(19) United States
(12) Patent Application Publication
(10) Pub. No.: US 2007/0250155 A1
(43) Pub. Date: Oct. 25, 2007

(54) Bioabsorbable medical device

(75) Inventor: John A. Simpson, Carlsbad, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Advanced Cardiovascular Systems, Inc.

(21) Appl. No.: 11/410,544

(22) Filed: Apr. 24, 2006

(51) Int. Cl. A61F 2/90

(52) U.S. Cl. 623/1.16; 623/1.38; 148/519; 148/321

(57) ABSTRACT

An implantable medical device is provided that degrades upon contact with body fluids so as to limit its residence time within the body. The device is formed of an iron carbon alloy that is subjected to DET heat treatment to impart high strength and high ductility in combination with an accelerated corrosion rate.

### BACKGROUND OF THE INVENTION

The present invention relates generally to medical devices which are adapted for implantation into a patient's body lumen and which are intended to gradually become absorbed by the body after implantation. More particularly, the invention is applicable to a stent for deployment in a blood vessel in which its presence is only temporarily required but initial high yield strength and good ductility is nonetheless needed.

Various medical devices are routinely implanted in a body lumen such as a blood vessel, wherein a permanent presence is not required and wherein an extended presence may actually be counterproductive. For example, stents are particularly useful in the treatment and repair of blood vessels after a stenosis has been compressed by percutaneous transluminal coronary angioplasty (PTCA), percutaneous transluminal angioplasty (PTA), or removed by atherectomy or other means, to help improve the results of the procedure and maintain patency. Alternatively, stents can be used to provide primary compression to a stenosis in cases in which no initial PTCA or PTA procedure is performed. It has however been found that the support that is provided by a stent is only required for a limited period of time, perhaps on the order of months, as the part of the vessel affected by stenosis would thereafter typically remain open even without any further support. The continued presence of some stent structures would then only serve as a permanent irritation of the tissue surrounding the stent, as the stent's rigidity could preclude it from performing the flexions caused by the heartbeat. An additional complication arises in pediatric applications because the stent comprises a fixed obstruction at the implantation site while such implantation site evolves with the growth of the child. Invasive retrieval of a stent is generally not considered to be a viable option.

While stents have typically been constructed of relatively inert metals in order to ensure their longevity, degradable stent structures have more recently been devised in an effort to provide support for only a limited period of time. Various polymeric substances are known that gradually dissolve and are absorbed by the body without adverse effect which has prompted the construction of stents with such polymers and polymer combinations for the purpose of providing only temporary support. It is however difficult to match the structural and mechanical properties of a metallic structure with the use of polymers, especially when polymeric materials are loaded with a drug, as drug loading of a polymeric material can have a significant adverse effect on strength. The need to minimize delivery profile as well as the desire to minimize bulk upon deployment substantially precludes simply increasing the dimensions of a polymeric stent in an effort to match the strength of a metallic structure.

It has more recently been found that certain metals, such as iron, are readily absorbable by the body without adverse effect. It has been shown in animal studies that bioabsorbable cardiovascular stents made from pure (>99.8%) iron do not cause local or systemic toxicity, do not induce significant neointimal proliferation, possess low thrombogenicity, and cause only mild inflammatory response of the stented vessel. Consequently, the use of iron is being considered for use in degradable stent applications. However, the yield strength of annealed pure iron is substantially less than the stainless steel (e.g. 316) and cobalt chrome (e.g. L605) alloys that have been found to be ideally suited for permanent stenting applications. High yield strengths are generally desirable for stent materials as they allow for thinner struts which translates into smaller crimped stent profiles. Unfortunately, the alloying elements that have been previously relied upon to realize gains in yield strength also serve to impart excellent corrosion resistance which would frustrate an effort to provide a bioabsorbable medical device.

A material is therefore needed with which a medical device can be fabricated, which is bioabsorbable, which has a high yield strength and which is ductile. It is therefore most desirable to raise the yield strength of iron without increasing corrosion resistance and without decreasing its ductility so as to be able to provide a bioabsorable stent.

### SUMMARY OF THE INVENTION

The present invention provides a medical device fabricated of iron in a form that is bioabsorbable yet has high yield strength and ductility. Such characteristics render the material especially well suited for use in stent applications. It has been found that the yield strength of iron can be substantially increased, without compromising ductility and without enhancing corrosion resistance with the inclusion of carbon and by subjecting the alloy to a heat treatment technique known as divorced-eutectoid-transformation (DET).

In accordance with the present invention, the carbon content of the Fe—C alloy is selected in a range from about 1% to 2.1% by weight. While such carbon content in steels subjected to conventional heat treatment methods would cause a brittle, intergranular network of iron carbide to form that drastically reduces ductility upon cooling, the DET heat treatment results in a spheroidized microstructure, wherein spherical particles of iron carbide (Fe3C) are surrounded by a matrix of essentially pure iron. As carbon content is increased, the volume percentage of iron carbide particles rises accordingly. The iron carbide particles naturally raise the yield strength by dispersion strengthening, and also provide unusually fine grain sizes by preventing grain growth during DET processing. Such microstructure results in a strong and ductile material.

While conventional heat treatment methods for steels with carbon contents above 0.8% typically cause formation of a brittle, intergranular network of iron carbide (a.k.a. “proeutectoid cementite”) that drastically reduces their ductility upon cooling to room temperature, a DET treatment allows the iron carbide in high carbon steel containing from 1.0 to 2.1% carbon to develop a strong and ductile microstructure. For example, an alloy of Fe/1.9%C subjected to DET processing can produce ferrite grain sizes in the range of 1 to 10 microns such that the yield strengths on the order of 800 MPa with a tensile strength of 1035 MPa and a 20% elongation are achievable. While such yield and strength values are comparable to those of the cobalt chrome used in stent applications, the elongation is considerably lower, albeit sufficient for such application.

A key advantage of using carbon as the only principal alloying element in pure iron, coupled with the DET processing method to produce a fine dispersion of iron carbides within a fine-grained ferrite matrix, is that its corrosion resistance remains unimproved and potentially somewhat diminished. Thus, the natural bioabsorbability of iron is not in any way compromised by alloying. Additionally, iron carbides act to stabilize the grain size during annealing treatments, thereby assuring that tubing made therefrom for fabricating stents would have a uniform, fine grain size. Fine grain size is important not only for improving yield strength, but also for enhancing fatigue resistance which is important in many medical device applications. Finally, alloying simply with carbon would not be expected to adversely affect an iron stent's local or systemic toxicity behavior, thrombogenicity, inflammatory response, etc.

The fabrication of a stent in accordance with the present invention would first call for the iron-carbon alloy to be formed into tubing such as by extrusion. The tubing is then subjected to the DET heat treatment process, followed by a drawing step or steps to reduce the tubing to the desired final dimensions. Conventional laser cutting may then be relied upon to create a stent pattern in the tubing followed by an electropolishing step. Due to the desired lack of corrosion resistance of the final product, greater care during the subsequent handling and packaging is required in order to prevent premature onset of the corrosion process.

Other features and advantages of the present invention will become more apparent from the following detailed description when taken in conjunction with the accompanying exemplary drawings.

### BRIEF DESCRIPTION OF THE DRAWINGS

FIG. 1 is an elevational view, partially in section, of a stent embodying features of the invention which is mounted on a delivery catheter and disposed within a damaged artery.

FIG. 2 is an elevational view, partially in section, similar to that shown in FIG. 1 wherein the stent is expanded within a damaged artery.

FIG. 3 is an elevational view, partially in section, depicting the expanded stent within the artery after withdrawal of the delivery catheter.

### DETAILED DESCRIPTION OF THE PREFERRED EMBODIMENTS

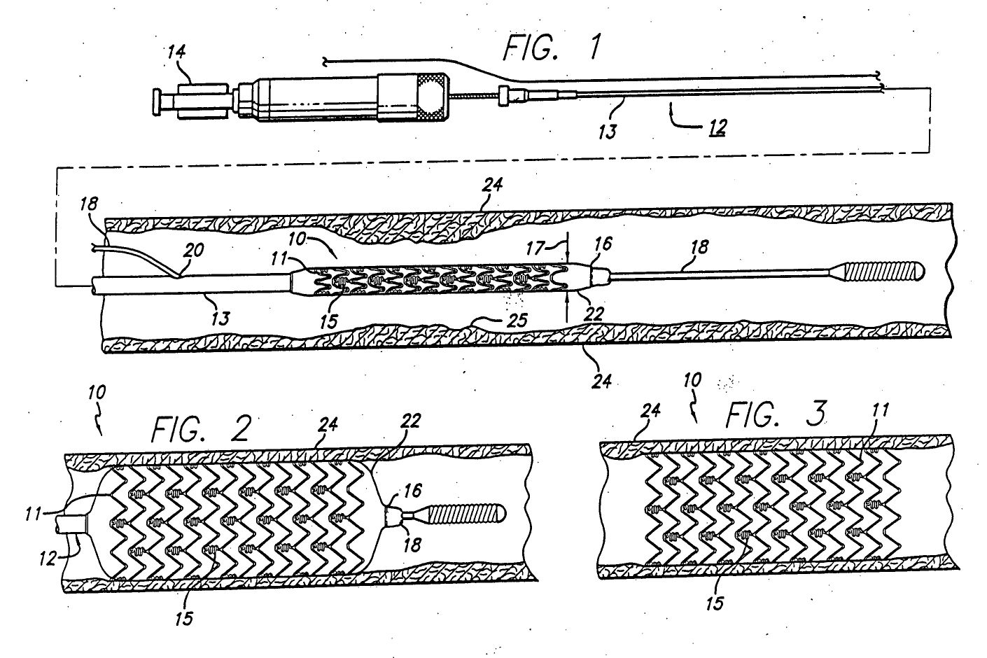

FIG. 1 generally depicts a corrodible metal stent 10, incorporating features of the invention, mounted on a catheter assembly 12 which is used to deliver the stent and implant it in a body lumen, such as a coronary artery, carotid artery, peripheral artery, or other vessel or lumen within the body. The stent generally comprises a plurality of radially expandable cylindrical rings 11 disposed generally coaxially and interconnected by undulating links 15 disposed between adjacent cylindrical elements. The catheter assembly includes a catheter shaft 13 which has a proximal end 14 and a distal end 16. The catheter assembly is configured to advance through the patient's vascular system by advancing over a guide wire by any of the well known methods of an over the wire system (not shown) or a well known rapid exchange catheter system, such as the one shown in FIG. 1.

Catheter assembly 12 as depicted in FIG. 1 is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries. Alternatively, the invention may be practiced using a self-expanding stent configuration as is well known in the art.

As shown in FIG. 1, a partial cross-section of an artery 24 is shown with a small amount of plaque that has been previously treated by an angioplasty or other repair procedure. Stent 10 of the present invention is used to repair a diseased or damaged arterial wall which may include the plaque 25 as shown in FIG. 1, or a dissection, or a flap which are commonly found in the coronary arteries, carotid arteries, peripheral arteries and other vessels.

In a typical procedure to implant stent 10, the guide wire 18 is advanced through the patient's vascular system by well known methods so that the distal end of the guide wire is advanced past the plaque or diseased area 25. Prior to implanting the stent, the cardiologist may wish to perform an angioplasty procedure or other procedure (e.g., atherectomy) in order to open the vessel and remodel the diseased area. Thereafter, the stent delivery catheter assembly 12 is advanced over the guide wire so that the stent is positioned in the target area. The expandable member or balloon 22 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIGS. 2 and 3, the balloon is fully inflated with the stent expanded and pressed against the vessel wall, and in FIG. 3, the implanted stent remains in the vessel after the balloon has been deflated and the catheter assembly and guide wire have been withdrawn from the patient.

The stent 10 serves to hold open the artery 24 after the catheter is withdrawn, as illustrated by FIG. 3. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with endothelial cell growth which further minimizes blood flow interference. The undulating portion of the stent provides good tacking characteristics to prevent stent movement within the artery. Furthermore, the closely spaced cylindrical elements at regular intervals provide uniform support for the wall of the artery, and consequently are well adapted to tack up and hold in place small flaps or dissections in the wall of the artery, as illustrated in FIGS. 2 and 3. The stent patterns shown in FIGS. 1-3 are for illustration purposes only and can vary in size and shape to accommodate different vessels or body lumens.

The stent illustrated in FIGS. 1-3 is formed of an iron carbon alloy and processed in accordance with the present invention. The iron carbon alloy preferably contains between 1.0 and 2.1% carbon, thereby classifying the alloy as an “ultra-high carbon steel”. Most preferably, the carbon content comprises 1.8%. Such alloy can be melted and cast as a conventional ingot or processed into a billet by powder metallurgy techniques.

The ingot or billet is then extruded into a tube or rod, wherein the latter is subsequently drilled to produce a hollow redraw blank. The high temperature deformation associated with extrusion not only provides for an efficient means for size reduction, but also serves to break up coarse, non-spheroidal carbides from the original ingot or billet. Post-extrusion heat treatment is then relied upon to induce divorced eutectoid transformation (DET) and thus create ultra-fine spheroidized carbides with a fine ferrite grain structure as is described in U.S. Pat. No. 4,448,613 which is incorporated herein in its entirety. The preferred DET process entails reheating the extrusion to above the eutectoid transformation temperature (about 780° C.) for about one hour such that pearlite is mostly dissolved into austenite in which the carbon is not uniformly distributed. The austenite will have a fine grain size because grain growth is inhibited by the presence of the spheroidized pro-eutectoid carbides. The extrusion is then air cooled below the eutectoid transformation temperature to produce a structure of fully spheroidized cementite in a fine ferrite matrix. The time and temperature that the alloy is held above the eutectoid transformation temperature and the precise composition of the alloy is of importance in attaining the fine, spheroidized structure. The exact soaking time (ranging from minutes to hours) depends on the product, size, shape, temperature (as the temperature is increased, the soaking time is decreased), and alloying elements present. For any specific new alloying element, only a few preliminary tests, obvious to those skilled in the art, need to be done to determine the correct time and temperature conditions for obtaining the desired fine-grained spheroidized structure.

The physical properties of the resulting material compares favorably to stainless steel alloys and cobalt chrome alloys that have heretofore been used in the fabrication of stents:

| Alloy | 0.2% Yield Strength | Ultimate Tensile Strength | Elongation |
|---|---|---|---|
| 316L | 366 MPa (53 ksi) | 675 MPa (98 ksi) | 43% |
| L605 | 629 MPa (91 ksi) | 1147 MPa (166 ksi) | 46% |
| Fe/1.8% C | 800 MPa (116 ksi) | 1035 MPa (150 ksi) | 20% |

While the strength of the iron carbide material of the present invention is well suited for use in stent applications, its corrosion rate is substantially undiminished from that of pure iron and thus subjected to accelerated degradation upon implantation in the human body. Moreover, alloying simply with carbon is not expected to adversely affect an iron stent's bioabsorbability, local or systemic toxicity behavior, thrombogenicity or inflammatory response. Alternatively, very minor amounts of other elements such as manganese or silicon may be added, in the absence of any toxic indications, both of which are commonly added to steels for deoxidation or to tie up trace amounts of sulphur.

After heat treatment, conventional tube drawing processes are used to reduce the tubing to the desired final dimensions for stent cutting. To avoid inadvertently converting the fine spheroidized microstructure into lamellar pearlite via ordinary eutectoid transformation, all annealing steps must be performed below about 725° C. This temperature restriction has the advantage of limiting the possibility of ferrite grain growth.

After the tubing has attained the desired final dimensions, a desired stent pattern may be cut using well known laser cutting techniques followed by an electropolishing step. A chemical passivation step is not required, since iron and low alloy steels generally do not passivate. Due to iron's relative lack of corrosion resistance, greater care must of course be taken during processing and packaging of the final stent product.

While the invention has been described in connection with certain disclosed embodiments, it is not intended to limit the scope of the invention to the particular forms set forth, but, on the contrary it is intended to cover all such alternatives, modifications, and equivalents as may be included in the spirit and scope of the invention as defmed by the appended claims. More particularly, a stent according to the present invention may be coated with one or more coatings whose primary function is to elude one or more drugs. Such drugs are commonly used to inhibit proliferation of endothelial cells and thus prevent restenosis, or to inhibit thrombus formation and thus prevent embolization. The coating or coatings would preferably be bioabsorbable, so that no significant residue remains after the underlying stent has been fully resorbed. Furthermore, the invention is readily applicable to any implantable medical device requiring bioabsorbability, high yield strength and good ductility.

What is claimed:

1. An implantable, bioabsorbable medical device formed of an alloy of iron and carbon, wherein the carbon substantially exists in the form of spheroidized iron carbide.

2. The implantable, bioabsorbable medical device of claim 1, wherein said alloy has a carbon content between 1.0% and 2.1%.

3. The implantable, bioabsorbable medical device of claim 2, wherein said alloy has a carbon content of about 1.8%.

4. The implantable bioabsorbable medical device of claim 1, having a grain size in the range of 1 to 10 microns.

5. The implantable bioabsorbable medical device of claim 1, comprising a stent.

6. The implantable bioabsorbable medical device of claim 1, comprising a stent, wherein said alloy has a carbon content of about 1.8% and a grain size of between 1 to 10 microns.

7. The implantable bioabsorbable medical device of claim 1, formed of a divorced-eutectoid-transformed alloy of iron and carbon.

8. The implantable bioabsorbable medical device of claim 7, wherein said iron alloy has a carbon content of between 1.0% and 2.1%.

9. The implantable bioabsorbable medical device of claim 8, wherein said carbon content comprises about 1.8%.

10. The implantable bioabsorbable medical device of claim 7 wherein said iron alloy has a grain size of in the range of 1 to 10 microns.

11. A method of forming a stent, comprising:
forming a tubular structure of an iron and carbon alloy,
heat treating said tubular structure such that the alloy undergoes divorced-eutectoid-transformation;
drawing said tubular structure to a desired dimension; and
laser cutting a stent pattern into drawn tubular structure.

12. The method of claim 11, wherein said tubular structure is formed by extrusion.

13. The method of claim 11, wherein said drawn tubular structure is subjected to annealing steps, all of which are performed below about 725° C.

14. The method of claim 11, wherein said heat treatment comprises:
heating said tubular structure to above about 780° C. for about one hour; and
air cooling said tubular structure to a temperature below about 780° C.
